(19) United States
(12) Patent Application Publication
(10) Pub. No.: US 2020/0038318 A1
(43) Pub. Date: Feb. 6, 2020

(54) ORAL MEDICATION FORMULATIONS

(71) Applicant: GRUNBIOTICS PTY LTD, Eltham, Victoria (AU)

(72) Inventors: Jamie HUGHES, Eltham, Victoria (AU); Cris POPP, Eltham, Victoria (AU); Bradley WARDROP-BROWN, Eltham, Victoria (AU)

(21) Appl. No.: 16/341,182

(22) PCT Filed: Oct. 20, 2017
(86) PCT No.: PCT/AU2017/051149

§ 371 Date: Apr. 11, 2019

(30) Foreign Application Priority Data
Oct. 20, 2016 (AU) 2016904267
Nov. 14, 2016 (AU) 2016904621
Aug. 11, 2017 (AU) 2017903222

(51) Int. Cl. A61K 9/46; A61K 31/519; A61K 9/00; A61K 31/714; A61K 31/4415; A61K 47/26; A61K 47/36; A61K 47/02; A61K 47/12

(52) U.S. Cl. CPC A61K 9/0007; A61K 31/519; A61K 9/0095; A61K 31/714; A61K 47/12; A61K 47/26; A61K 47/36; A61K 47/02; A61K 31/4415

(57) ABSTRACT

A preparation comprising an active ingredient, a suspension base and an effervescent agent wherein the active ingredient comprises one or more B vitamins. The suspension base comprises at least one of inulin, dextrins such as maltodextrin, cellulose derivatives, gums such as xanthan gum, and other hydrocolloids; preferably a combination of inulin and xanthan gum.

### CROSS-REFERENCE TO RELATED APPLICATION

The present patent application is US National stage application PCT/AU2017/051149 filed Oct. 20, 2017, which claims priority to three Australian patent applications: AU 2016904267 filed Oct. 20, 2016, AU2016904621 filed Nov. 14, 2016 and AU2017903222 filed Aug. 11, 2017, all of which incorporated herein by reference in their entirety.

### BACKGROUND OF THE INVENTION

With an ageing population, and increasing survival rate for many injuries and illnesses, there are increasing number of people with chronic health conditions. According to the National Medical Expenditure Survey of 1987, 90 million Americans suffer from one or more chronic conditions. Treatment of these chronic conditions represents over 76% of health care expenditures and the total direct costs of treating these chronic conditions is estimated to rise to $798 billion by the year 2030.

Chronic health conditions are conditions that persist over a long period of time and that may or may not be cured. In such circumstances, the sufferer is required to maintain a treatment regime for many months and years. Many patients with chronic conditions are treated at home. Unfortunately, many patients may be unable to reliably manage their treatment, at home without the supervision of a healthcare provider.

The reference to any prior art in this specification is not, and should not be taken as, an acknowledgement or any form of suggestion that the prior art forms part of the common general knowledge.

### SUMMARY OF THE INVENTION

The present invention relates to the use of effervescent formulations of medication, medical foods, or supplements, or some combination of these for example to reduced treatment interruptions, and increase treatment compliance for patients and consumers with chronic health conditions.

The invention provides a preparation comprising one or more active ingredients, a suspension base and an effervescent agent wherein the active ingredient comprises one or more B vitamins. The suspension base may comprise a matrix-forming component and a thickening (or viscosity) component wherein optionally the thickening/viscosity component is provided by the presence of one or more hydrocolloids. The matrix forming component may comprise one or more of: inulin, dextrins, malto dextrins, potato dextrins. Preferably the matrix forming component may comprise inulin.

The thickening/viscosity component may comprise one or more of: Xanthum gum, carboxymethyl cellulose, a hydratable methyl cellulose, sodium carboxyl methyl cellulose, sodium methyl cellulose, microcrystalline cellulose group, other gums such as guar gum, pectin, or other hydrocolloids, agar, carrageenan. In some embodiments, the thickening (or viscosity) component comprises xanthum and preferably prehydrated xanthum.

In some embodiments the effervescence produced assists in mixing of the ingredients of the preparation after water has been added and thereby enhances formation of a suspension.

The invention also provides a product for dilution to make a liquid serve comprising for example optionally one or more of about 5 to 40 mg, optionally 10 to 30 mg and optionally about 15-20 mg of active ingredient per 200 ml serve.

The phrase “a physiologically acceptable salt, ester or derivative thereof” as used herein refers to various salts, esters, or derivatives of the molecule referred to which would be understood to be physiologically acceptable by the skilled addressee such that one or more of them (and for example a plurality of them in combination) could be used place of the named molecule.

The invention also provides composition comprising:

0.1-100 mg optionally 1 to 80 mg, optionally 10 to 50 mg, optionally 12 to 25 mg, optionally 10 to 20 mg L-methylfolate or a physiologically acceptable salt, ester or derivative thereof;
0.1 to 10 mg optionally 0.3 to 8 mg, optionally 0.5 to 5 mg, optionally 0.5 to 2 mg, optionally 0.6 to 1.2 mg Cyanocobalamin or a physiologically acceptable salt, ester or derivative thereof;
10 to 1000 mg optionally 20 to 800 mg optionally 40 to 400 mg optionally 80 to 300 mg, optionally 120 to 200 mg flavour additive which is optionally a natural flavour additive and optionally pink grapefruit flavour or the like;
80 to 8000 mg optionally 200 to 4000 mg optionally 400 to 2000 mg, optionally 600 to 1000 mg optionally 750 to 900 mg inulin which is optionally instant inulin-fibriline;
3 to 300 mg optionally 10 to 200 mg, optionally 15 to 100 mg, optionally 25 to 50 mg, optionally 25 to 35 mg sucralose;
80 to 8000 mg optionally 200 to 4000 mg optionally 400 to 2000 mg, optionally 600 to 1000 mg optionally 750 to 900 mg glucose syrup which is optionally glucose solids—rice;
40 to 5000 mg optionally 80 to 3000 mg, optionally 100 to 1000 mg, optionally 200 to 800 mg, optionally 400 to 600 mg sodium carbonate;
150 to 15,000 mg optionally 300 to 10,000 mg, optionally 500 to 5000 mg, optionally 1000 to 2000 mg, optionally 1200 to 1700 mg citric acid anhydrous;
1 to 200 mg optionally 1 to 100 mg optionally 1 to 50 mg, optionally 1 to 30 mg, optionally 10 to 22 mg natural colour;
1 to 200 mg optionally 1 to 100 mg optionally 1 to 50 mg, optionally 1 to 30 mg, optionally 10 to 22 mg xanthan gum 200 mesh;
40 to 5000 mg optionally 80 to 3000 mg, optionally 100 to 1000 mg, optionally 200 to 800 mg, optionally 450 to 650 mg sodium bicarbonate.

The invention also provides a composition comprising:

0.1 to 10 mg optionally 0.3 to 8 mg, optionally 0.5 to 5 mg, optionally 0.8 to 4 mg, optionally 1.5 to 3 mg L-methylfolate or a physiologically acceptable salt, ester or derivative thereof;
0.1 to 10 mg optionally 0.3 to 8 mg, optionally 0.5 to 5 mg, optionally 0.9 to 3.5 mg, optionally 1.5 to 3 mg Cyanocobalamin or a physiologically acceptable salt, ester or derivative thereof;
0.1-200 mg optionally 1 to 120 mg, optionally 10 to 100 mg, optionally 20 to 80 mg, optionally 40 to 60 mg Vitamin B6 or a physiologically acceptable salt, ester or derivative thereof;
50 to 15,000 mg optionally 150 to 10,000 mg, optionally 500 to 5000 mg, optionally 1000 to 2000 mg, optionally 1500 to 2500 mg omega three fatty acid which optionally comprises docosahexaenoic acid (DHA) or eicosapentaenoic acid (EPA) or a mixture thereof;
10 to 1000 mg optionally 20 to 800 mg optionally 40 to 400 mg optionally 80 to 300 mg, optionally 120 to 200 mg flavour additive which is optionally a natural flavour additive and optionally pink grapefruit flavour or the like;
80 to 8000 mg optionally 200 to 4000 mg more optionally 400 to 2000 mg, optionally 600 to 1000 mg optionally 750 to 900 mg inulin which is optionally instant inulin-fibriline;
3 to 300 mg optionally 10 to 200 mg, optionally 15 to 100 mg, optionally 25 to 50 mg, optionally 25 to 35 mg sucralose;
80 to 8000 mg optionally 200 to 4000 mg more optionally 400 to 2000 mg, preferably 600 to 1000 mg optionally 750 to 900 mg glucose syrup which is optionally glucose solids—rice;
40 to 5000 mg optionally 80 to 3000 mg, optionally 100 to 1000 mg, optionally 200 to 800 mg, optionally 400 to 600 mg sodium carbonate;
150 to 15,000 mg optionally 300 to 10,000 mg, optionally 500 to 5000 mg, optionally 1000 to 2000 mg, optionally 1200 to 1700 mg citric acid anhydrous;
1 to 200 mg optionally 1 to 100 mg optionally 1 to 50 mg, optionally 1 to 30 mg, optionally 10 to 22 mg natural colour;
1 to 200 mg optionally 1 to 100 mg optionally 1 to 50 mg, optionally 1 to 30 mg, optionally 10 to 22 mg xanthan gum 200 mesh;
40 to 5000 mg optionally 80 to 3000 mg, optionally 100 to 1000 mg, optionally 200 to 800 mg, optionally 450 to 650 mg sodium bicarbonate.

In some preferred embodiments of this composition, there is provided 0.1 to 2 mg, optionally 0.2 to 1.5 mg optionally 0.5 to 1.3 mg optionally 0.6 to 1.2 mg, optionally 0.7 to 1.1 mg, optionally 0.8 to 1.0 mg, optionally 0.89 mg Vitamin B6 or a physiologically acceptable salt, ester or derivative thereof.

In some preferred embodiments of this composition, there is provided 35 to 55 mg, optionally 40 to 48 mg, optionally 42 to 46 mg, optionally 43 to 45 mg, optionally 44.1 mg, optionally 20 mg, Vitamin B6 or a physiologically acceptable salt, ester or derivative thereof.

In some preferred embodiments of this composition, there is provided 0.001 to 0.02 mg, optionally 0.002 to 0.015 mg optionally 0.005 to 0.013 mg optionally 0.006 to 0.012 mg, optionally 0.007 to 0.011 mg, optionally 0.008 to 0.01 mg, optionally 0.0089 mg Cyanocobalamin or a physiologically acceptable salt, ester or derivative thereof.

In some preferred embodiments of this composition, there is provided 0.5 to 7 mg optionally 1 to 5 mg, optionally 2 to 3 mg, optionally 2.1 to 2.4 mg, optionally 2.21 mg, optionally 0.5 mg Cyanocobalamin or a physiologically acceptable salt, ester or derivative thereof.

In some preferred embodiments of this composition, there is provided docosahexaenoic acid (DHA) in an amount chosen from 100 or 200 or 300 or 400 or 500 or 600 or 700 mg.

In some preferred embodiments of this composition, there is provided L-methylfolate or a physiologically acceptable salt, ester or derivative thereof in an amount chosen from 1.1 or 2 or 3 or 4 or 5 or 6 or 7 or 8 mg.

The invention also provides a composition comprising:

0.1-100 mg optionally 1 to 80 mg, optionally 10 to 50 mg, optionally 12 to 25 mg, optionally 10 to 20 mg L-methylfolate or a physiologically acceptable salt, ester or derivative thereof;
10 to 1000 mg optionally 20 to 800 mg optionally 40 to 400 mg optionally 80 to 300 mg, optionally 120 to 200 mg flavour additive which is optionally a natural flavour additive and optionally pink grapefruit flavour or the like;
80 to 8000 mg optionally 200 to 4000 mg optionally 400 to 2000 mg, optionally 600 to 1000 mg optionally 750 to 900 mg inulin which is optionally instant inulin-fibriline;
3 to 300 mg optionally 10 to 200 mg, optionally 15 to 100 mg, optionally 25 to 50 mg, optionally 25 to 35 mg sucralose;
80 to 8000 mg optionally 200 to 4000 mg optionally 400 to 2000 mg, optionally 600 to 1000 mg optionally 750 to 900 mg glucose syrup which is optionally glucose solids—rice;
40 to 5000 mg optionally 80 to 3000 mg, optionally 100 to 1000 mg, optionally 200 to 800 mg, optionally 400 to 600 mg sodium carbonate;
150 to 15,000 mg optionally 300 to 10,000 mg, optionally 500 to 5000 mg, optionally 1000 to 2000 mg, optionally 1200 to 1700 mg citric acid anhydrous;
1 to 200 mg optionally 1 to 100 mg optionally 1 to 50 mg, optionally 1 to 30 mg, optionally 10 to 22 mg natural colour;
1 to 200 mg optionally 1 to 100 mg optionally 1 to 50 mg, optionally 1 to 30 mg, optionally 10 to 22 mg xanthan gum 200 mesh;
40 to 5000 mg optionally 80 to 3000 mg, optionally 100 to 1000 mg, optionally 200 to 800 mg, optionally 450 to 650 mg sodium bicarbonate.

The invention also provides a method of ameliorating or reducing the clinical signs of a brain-related condition comprising administering a preparation or composition according to the invention.

The invention also provides an effervescent formulation comprising at least one active agent, at least one pharmaceutically acceptable effervescent acid, at least another effervescent base and at least another excipient wherein the active agent comprises a B vitamin and optionally a folate or a folate derivative.

The invention provides a formulation comprising a B vitamin characterized in that said formulation comprises an effervescent couple which is composed of at least one effervescent acid and at least one effervescent base and at least one pharmaceutically acceptable excipient.

The invention provides a product comprising: one or more natural isomers of reduced folate selected from the group consisting of (6S)-tetrahydrofolic acid, 5-methyl-(6S)-tetrahydrofolic acid, 5-formyl-(6S)-tetrahydrofolic acid, 10-formyl-(6R)-tetrahydrofolic acid, 5,10-methylene-(6R)-tetrahydrofolic acid, 5,10-methenyl-(6R)-tetrahydrofolic acid, 5-formimino-(6S)-tetrahydrofolic acid, and polyglutamyl derivatives thereof; and a fizz composition including acid and base components, wherein the fizz composition reacts to create fizzing in the food product when combined with an aqueous liquid.

The present disclosure provides for the novel combination of effervescence with medications that need to be taken for chronic health conditions. The preparations of the invention are able to make the medication easier and more desirable to take, and to improve treatment compliance by reducing treatment interruptions

In one aspect of the invention there is provided a preparation comprising an active ingredient, a suspension base and an effervescent agent. It has been found that preparations according to the invention are particularly preferred for active ingredients that are poorly soluble. One example of such an ingredient is the calcium salt of L-methyfolate. Preferably the suspension base comprises a matrix-forming component and a thickening (or viscosity) component. In some preferred embodiments, the thickening/viscosity component is provided by the presence of one or more hydrocolloids.

In one aspect of the invention, there is provided an effervescent formulation comprising at least one active agent, at least one pharmaceutically acceptable effervescent acid, at least another effervescent base and at least another excipient. In some preferred embodiments, the active agent(s) comprise a vitamin B and in some embodiments it comprises a folate or a folate derivative.

In another aspect of the invention, there is provided a formulation comprising a vitamin B (such as a folate or folate derivative), characterized in that said formulation comprises an effervescent couple which is composed of at least one effervescent acid and at least one effervescent base and at least one pharmaceutically acceptable excipient and is in effervescent form.

In another aspect of the invention there is provided a food, food special medical purposes, medication, supplement taken from chronic health condition that has a fizz component. Another aspect of the invention comprises a food, supplement, a food special medical purposes, medication or medical food that has a fizz component.

In one embodiment, the present invention relates to a composition which includes one or more natural isomers of reduced folate. The one or more natural isomers of reduced folate is selected from the group consisting of (6S)-tetrahydrofolic acid, 5-methyl-(6S)-tetrahydrofolic acid, 5-formyl-(6S)-tetrahydrofolic acid, 10-formyl-(6R)-tetrahydrofolic acid, 5,10-methylene-(6R)-tetrahydrofolic acid, 5,10-methenyl-(6R)-tetrahydrofolic acid, 5-formimino-(6S)-tetrahydrofolic acid, and polyglutamyl derivatives thereof.

In some embodiments, the invention provides a novel combination of effervescence with 1-methylfolate in all its forms and isomers taken as a medical food, food special medical purpose, medication, supplement for a variety conditions, including but not limited to Alzheimer's dementia, depression, neural tube defects, diabetes and other conditions. The purpose of this combination is to make the medication easier and more desirable to take, and to improve treatment compliance by reducing treatment interruptions.

In one aspect the invention provides a product comprising:

a medical food, a food for special medical purpose, a nutritional supplement, pharmacological product, nutraceutical, dietary composition or any compound, powder, herb, or treatment for chronic health conditions and a fizz composition including acid and base components, wherein the fizz composition reacts to create fizzing in the food product when combined with an aqueous liquid.

In some embodiments the invention further comprises a substance taken to support, prevent, treat or address a disease state, including supporting the nutritional deficiencies associated with that disease state, reducing or managing the symptoms of disease, curing the disease, or dealing with side effects of the disease.

As used herein, an effervescent compound is any compound that effervesces when mixed with a liquid—regardless of the chemical process. As used herein, a treatment regime means the consumption of the compound, in the dosage recommended or prescribed, and at the frequency and times required. As used herein, a recommended or required medication might be by the prescribing medical practitioner, or the manufacturer, or some other health practitioner. As used herein, a medical practitioner is a health professional, including a doctor, naturopath, chemist, pharmacist, occupational therapist, nutritionist, dietician, gerontologist, psychologist, psychiatrist, specialist, therapist, or some other health professional. A supplement as used herein may be a powder, tablet, capsule or take some other form. The skilled addressee will appreciate that a product according to the invention may contain other ingredients not listed herein.

In another aspect, the invention provides a product comprising: one or more natural isomers of reduced folate selected from the group consisting of (6S)-tetrahydrofolic acid, 5-methyl-(6S)-tetrahydrofolic acid, 5-formyl-(6S)-tetrahydrofolic acid, 10-formyl-(6R)-tetrahydrofolic acid, 5,10-methylene-(6R)-tetrahydrofolic acid, 5,10-methenyl-(6R)-tetrahydrofolic acid, 5-formimino-(6S)-tetrahydrofolic acid, and polyglutamyl derivatives thereof; and a fizz composition including acid and base components, wherein the fizz composition reacts to create fizzing in the food product when combined with an aqueous liquid.

Throughout this specification (including any claims which follow), unless the context requires otherwise, the word ‘comprise’, and variations such as ‘comprises’ and ‘comprising’, will be understood to imply the inclusion of a stated integer or step or group of integers or steps but not the exclusion of any other integer or step or group of integers or steps.

### DETAILED DESCRIPTION OF EXEMPLARY EMBODIMENTS

It is convenient to describe the invention herein in relation to particularly preferred embodiments. However, the invention is applicable to a wide range of implementations and products and it is to be appreciated that other constructions and arrangements are also considered as falling within the scope of the invention. Various modifications, alterations, variations and or additions to the construction and arrangements described herein are also considered as falling within the ambit and scope of the present invention.

In one embodiment, the chronic condition may be related to a lack of one or more folates. Folates are ubiquitous to nearly all forms of life. Humans and many other animals lack the capacity to make their own folate which thus is an essential vitamin, one type of essential nutrient. Anemia especially during pregnancy and in the geriatric population was an early indication of a dietary requirement for folate. A major function of folate is to remove one-carbon units from molecules being metabolized and then deliver them to molecules being synthesized. As an example, folate participates in the formation of the nucleic acids. Further, the activity of DNA is controlled, in part, by methylation, and the primary methylating agent of the body (S-adenosylmethionine) is made in a metabolic cycle involving a folate. Many studies have, therefore, focused on the relationship of folate status to cancer susceptibility, especially colorectal adenoma.

Treatment regimes, whether for chronic conditions, in general, conditions where folate is indicated may include taking medication, taking supplements, taking medical foods, or foods for special medical purposes, sticking to a prescribed diet, and even exercise and other activities.

To be effective, medications, medical foods and supplements often must follow various scheduling and dietary guidelines. For example, some are to be taken with food, others are not. Some medications are to be taken only once a day, others multiple times per day.

Under such conditions, the sufferer has a daily burden, of not only remembering what medication/supplementation/food they need to take on any particular day, or time of day, but the unpleasant task of swallowing many different pills or capsules

Remembering when to take a medication, supplement or medical food and how much of it can become difficult as the number of concurrent medications/supplements and/or medical foods increases. Furthermore pills, capsules and powders are more difficult to swallow for the elderly, very young, and those for whom the factory/digestive system is compromised. Even when the patient wants to adhere to the regime. They may involuntarily fail to.

In addition, a phenomenon called “pill fatigue”, often develops in long-term consumers/patients. Pill fatigue is a phenomenon whereby consumers resist taking their medication, out of boredom, difficulty swallowing or period other reasons, or forget. The process of adhering to their medical regime becomes unpleasant or boring. Pill fatigue, or resistance to a treatment regime, combined with accidental, or in voluntary non-adherence results in a phenomenon known as “Treatment Interruptions” (TI).

Unplanned, or unforeseen TI results in lower compliance with treatment regimes. This has a number of consequences for the consumer/patient. First, the consumer is not getting the full benefit of the treatment regime. Many compounds need to be in the body at the required levels in order to be efficacious. Insufficient levels, on the medication, medical food or related substance may mean that the treatment is only partially effective—or not effective at all.

Secondly, and more dangerously, it is difficult for the medical practitioner to determine if the regime is right for the patient. This can result in a misdiagnosis wherein the practitioner assumes the regime is not working, when, in fact, the real issue is that the medication has not been taken in the correct dose. This could result in a higher dose, or switch to another drug. It could even lead to a misdiagnosis of the underlying condition.

Pharmaceutical companies have long been aware of the problem of TIs, and a number of technologies exist to help with this. Various devices for assisting patients to following treatment regimens are known. For example, U.S. Pat. No. 4,837,719 to McIntosh describes a medication clock for signaling the times that dosages of a medication should be taken. The McIntosh device also provides a record of when each medicine was taken for comparison with the medication schedule. In addition, the McIntosh device can monitor and record temperature, blood pressure and pulse rate of the user.

Existing approaches usually involve some form of monitoring of adherence to the drug regime, or an apparatus that administers the drug. Both of these help to deal with the issue of treatment interruptions through policing compliance. They are in effect a “stick” approach to compliance.

A better approach is to increase compliance by improving the “likeability” of the medication/compound. By making the compound more enjoyable to consume, or by making it a “fun” part of the treatment regime, adherence to the treatment regime increases voluntarily.

In one embodiment, the product comprises a medication, medical food, food for special medical purposes, and/or supplement product for a chronic health condition/s that has a fizz component. The product can be formed from various compounds, and may be prescription or non-prescription. Additional ingredients, such a as sweeteners, flavorings, colorings, vitamins, minerals, preservatives, and other compositions known to the skilled practitioner, can be added, as desired.

As described herein, a fizz component is a compound or combination of compounds that release gas to make the product fizz upon contact with aqueous liquid.

In one embodiment, the product comprises a medication, medical food, food for special medical purposes, and/or supplement product comprised of 1-methylfolate in one or more of its isomers combined with a fizz component.

Such product may be used to treat, manage the symptoms of, or support the nutritional needs of sufferers, with a wide range of conditions including but not limited to Alzheimer's dementia, depression, major depression, ADHD, a ADD, as Aspergers Spectrum Disorders (ASD) including autism, neurological disorders, diabetes, etc. The product may be prescription or non-prescription. Additional ingredients, such a as sweeteners, flavorings, colorings, vitamins, minerals, preservatives, and other compositions known to the skilled practitioner, can be added, as desired.

The product comprising a fizz component can be any substance that can exhibit a “fizz” or an effervescent effect. The products comprising a fizz component, all components, reactants, initiators, by-products, and reaction products should be edible or not be harmful to the consumer.

In accordance with one embodiment of the invention, the product comprises a fizz component. The fizz component reacts when subjected to liquid aqueous initiator to fizz or exhibit an effervescent effect. The liquid can be any liquid that initiates the fizz effect. Typically products are mixed with water, which can be a suitable initiator. Other aqueous liquids, such as milk, buttermilk, fruit juice, and yogurt also are useful. A combination of different liquids also is useful.

The liquid can be hot or cold, depending upon the requirements of the product. In one embodiment, the product reacts when subjected to hot liquid. The temperature of the hot liquid, in one embodiment, is close to or at its boiling point. Other temperatures may also useful. Any temperature that is able to initiate the reaction that causes the fizz or effervescent effect is suitable.

The fizz component typically comprises acid and base components. Any food grade combination of acid and base components that provides effervescence to the product essentially without adversely affecting the organoleptic properties and characteristics of the product is suitable. Thus, any acid/base pair that provides a release of gas from the base to “fizz” the products is suitably used. Typically, the acid will be a food grade acid having a concentration sufficient to react with a food grade base that liberates a gas, typically carbon dioxide (CO2), to provide the “fizz” or effervescent effect.

Any food grade acid may be used. The acid is selected to ensure that the effervescent effect is realized yet the flavor of the product is not adversely affected. In one embodiment, the acid component preferably comprises an anhydrous acid selected from the group consisting of tartaric, malic, fumaric, adipic, succinic, acetic, lactic, propionic, sorbic, phosphoric, and blends thereof. More preferably, the acid component comprises citric acid.

The base component comprises any food grade basic compound that releases a gas upon reaction with the acid component to product the fizz or the effervescent effect. The gas released is limited only by the need to produce an edible product. Thus, the smell of the gas should be pleasant or inoffensive. Carbon dioxide gas is a preferred gas.

In one embodiment, the base component is selected from the group consisting of carbonate or bicarbonate of sodium, potassium, calcium, ammonium, and blends thereof. Preferably, the base component comprises calcium carbonate, sodium bicarbonate, or a combination thereof.

The relative proportions of base component and acid component are established to ensure that the fizz or effervescent effect is achieved efficiently, without leaving excess base or acid component unreacted in the product. Unreacted base or acid component might adversely affect the flavor of the product. Typically, therefore, a stoichiometric quantity of acid and base is utilized with essentially no excess of either acid or base. This method is used to ensure that essentially all of the base and acid components have been consumed by the effervescence reaction. Any minor amount of either acid or base component that has not reacted thus will remain in the product, typically without adversely affecting the organoleptic properties and characteristics of the product.

In one embodiment, the fizz component comprises between about 0.01 and about 10 weight percent, typically between about 0.05 and about 5 weight percent of acid component, and between about 0.01 and about 10 weight percent, typically between about 0.05 and about 5 weight percent of base component, both based on the total weight of the product. Preferably, the fizz component comprises stoichiometric quantities of each component. Typically, therefore, the molar ratio of acid component to base component is about 1:1 for bicarbonates to about 2:1 for carbonates. The skilled practitioner will, with the guidance provided herein, be able to determine an appropriate molar ratio. Other compositional ranges may also be useful.

A two-part (base and acid) fizz component typically is prepared prior to being added to the product. Preparation of the fizz component includes, for example, grinding the acid and base components into particles or powder. The grinding should result in an even distribution of the components. Preferably, the components are prepared to reduce formation of precipitates. This may be achieved by grinding the acid and base components together. Grinding the acid and base components separately followed by blending to produce an even distribution may yield a suitable fizz component.

The particle size of the fizz composition components is selected to, for example, achieve the desired reaction rate. Generally, reaction rate and particle size are inversely proportional. Typically, particles have an average particle size such that 95 weight percent of the particles are 42 mesh or finer. For example, higher reaction rate is achieved with smaller particles. In one embodiment, the average particle size of the components is established so that 95 weight percent of the particles are 80 mesh or finer. Providing a fizz component with components having other particle sizes may also be useful. The rate and duration of the effervescent effect will be different, as the skilled practitioner recognizes.

The prepared fizz component can be added to the product in various forms. In one embodiment, the prepared fizz component is added in dry form. For example, the fizz component is added as powder in the desired amount. The fizz component then is blended with the other components to produce an even distribution. To prevent premature reaction between the acid and base before the addition of an aqueous liquid, the moisture level of the components should be sufficiently low. The moisture level, for example, should be less than about 15 weight percent, and preferably less than about 10 weight percent. Alternatively, the fizz component can be encapsulated. Fizz component that is encapsulated is precluded from reacting to form gas and exhibit an effervescent effect. Either component may be encapsulated to achieve this result. Often, however, as the acid component and the base component are mixed, especially before grinding, both components are encapsulated. However, care should be taken to ensure that the encapsulation is not breached during mixing of fizz components and other components.

The encapsulant can be any food grade water-resistant coating. Often, such coatings comprise fat-based compositions that are fluid at elevated temperature but solidify at ambient temperature (about 20° C.-about 30° C.: about 68° F.-about 86° F.). Because the coating is solid at ambient temperatures, the acid component and the base component are precluded from reacting. However, the coating is breached at an elevated temperature, i.e., at a temperature at which the product is prepared for consumption. Then, the acid and base components can combine to yield the effervescent effect, or the fizz.

Suitable coatings for the particulate include, for example, coatings described in U.S. Pat. No. 6,159,511, which is herein incorporated by reference. Various types of edible fat-based coating can be used. Such edible fat can include, for example, cocoa, butter, coconut oil, soybean, cottonseed, sunflower, canola, partially hydrogenated vegetable oil, and combinations thereof. These and other fats can be used to form the basis of a coating for the acid and base component particles. Sugar can be also added to the coating for flavoring. Another suitable coating is Mor-Rex 1918, a hydrolyzed cereal solid having a dextrose equivalence of 10 available from CPC Industrial.

Other coatings also can be used. Waxes often are soluble in warm fluid appropriate for preparation of the product. Also, a coating can comprise a water-absorbing polymer molecule interspersed in the coating. The water-absorbent polymer swells and disrupts the coating upon absorption of water. Then, the coating fails and the components can mix with each other.

To coat the acid and base components, a suitable coating is prepared and heated to become fluid. The acid and base components then are mixed into the fluid coating. The coating then is solidified by cooling and the coated, two-component mixture is used in the manufacture of a product. The encapsulated product of this embodiment then can be solidified in a manner known in the art. A solid slab could be broken up, but crushing or comminating the slab is difficult to do well, i.e., to produce reasonably sized encapsulated products. Thus, particles often are formed by spray cooler, to form smaller, more easily managed and used drops. With the guidance provided herein, the skilled practitioner will be able to form encapsulated solid particles of acid and base components for inclusion in the product.

In another embodiment, the fizz component is further processed to form pellets. The pellets can be added to the product in the desired amount. The processed pellets can be encased in a coating. In one embodiment, the coating comprises edible fat. The pellet coatings can include the material used to coat the individual particles, as described above. These coatings also can be include coating interrupters, as described, to ensure that the acid and base components react fully (and yield a complete “fizz” reaction).

The content of a fat-based coating is selected to have a desired melting point. When subjected to an aqueous liquid having a temperature greater than the melting point of the coating, the coating melts. The melt rate increases as the difference between the melting point and the liquid temperature increases. A water-soluble coating dissolves in the aqueous liquid. Dissolution rate typically is greater at higher liquid temperatures. This melting or dissolution of the coating exposes the fizz component to the aqueous liquid, resulting in the fizzing or effervescent effect.

In one embodiment, the coating melts at a predetermined temperature. The predetermined temperature, for example, is less than or equal to about the temperature of the hot liquid used to prepare the product. In one embodiment, the predetermined melting point temperature is at about that of the hot liquid used to prepare the product. When the coating has water-absorbing swelling aid, these water-based inclusions in the coating react upon wetting and tend to break up the coating. Hot water, i.e., at or above the melting temperature of the fat-based material, will tend to melt more of the coating. Typical temperatures are in the range 20° C. to 100° C., preferably 30° C. to 90° C., and more preferably 40° C. to 80° C.

The time required to expose the fizz component can be determined by the thickness of the coating. The thicker the coating, the greater the time needed. The thickness of the coating is selected so that the fizz component becomes exposed to the liquid in from about two seconds to about ten minutes. Depending on the desired application or product, the exposure time may be shorter or longer. For example, the exposure time may be selected to provide a fizzing product after it is prepared and ready for eating. The case of the coating would then be thicker for a non-instant type of product than for an instant product to enable the pellets to withstand the cooking time before being melted or dissolved. Alternatively, the coated pellets may be added after the food is prepared and ready to eat, allowing the use of a relatively thinner coating. Thus, with the guidance provided, the skilled practitioner can determine a suitable coating thickness.

The product comprises a sufficient amount of fizz component to cause fizzing or an effervescent effect when an aqueous liquid is added. Preferably, the fizz component is present in a sufficient amount to produce fizzing for up to about ten minutes, and more typically for between about five seconds and five minutes. The fizz also can persist during eating of the product to provide a desirable mouth feel (organoleptic) experience. For example, the duration time can be selected to be relatively short, providing initial attraction or entertainment but ceasing to fizz when about to be consumed. Similarly, coatings of various thicknesses can be applied to portions of the fizz component to provide fizzing or an effervescent effect for an extended period.

Preferably, the amount of fizz component is less than the quantity that would adversely impact the taste of the product. The taste can be adversely affected by the salt or other reaction products formed during the reaction, or by unreacted base acid or component. It may also be desirable to provide the fizz component at levels which do not impart a substantial degree of gas, particularly carbonation, to the aqueous liquid. In one embodiment, the fizz component comprises between about 0.01 weight percent and about 10 weight percent, more typically between about 0.05 weight percent and about 5 weight percent of the total weight of the product, excluding the aqueous liquid. Other percentages of fizz component may also useful.

In yet another embodiment, fizz component may be provided in a combination of different forms to the product. In another embodiment, the fizz component can be a carbonation source for hot products, such as, for example, instant or non-instant hot beverages.

The fizz or effervescent component of the invention may optionally comprise a pharmaceutically acceptable effervescent couple comprising an acid and a base, wherein said acid of said effervescent couple is selected from the group consisting of citric, tartaric, amalic, fumeric, adipic, and succinic acids, and said base of said effervescent couple is selected from the group consisting of sodium bicarbonate, sodium carbonate, potassium bicarbonate, potassium carbonate and magnesium carbonate.

An effervescent reaction typically consists of:

Acid+Carbonate Salt to produce CO2+Acid Salt+H₂O

For example: Citric Acid+3NaHCO3 to produce 3CO2+Na3Citrate+3H2O

Example carbonate salts are sodium carbonate (soda ash) Na2, CO3, and sodium bicarbonate (baking soda), 3 NaHCO₃

The acid component may be selected from the group consisting of citric, amalic, boric, succinic, tartaric, malic, amalic, fumaric, adipic, acetic, lactic, propionic, sorbic, phosphoric, acid salt, sodium hydrogen sulfate etc and blends thereof.

The Carbonate source material may be selected from the alkaline earth metal carbonate and bicarbonate of sodium, potassium, calcium, ammonium, magnesium etc and blends thereof.

An active compound of the dosage form of the invention may be selected from the group consisting of one or more natural isomers of reduced folate is selected from the group consisting of (6S)-tetrahydrofolic acid, 5-methyl-(6S)-tetrahydrofolic acid, 5-formyl-(6S)-tetrahydrofolic acid, 10-formyl-(6R)-tetrahydrofolic acid, 5,10-methylene-(6R)-tetrahydrofolic acid, 5,10-methenyl-(6R)-tetrahydrofolic acid, 5-formimino-(6S)-tetrahydrofolic acid, and polyglutamyl derivatives thereof.

Other active compounds may also be selected from vitamin B6, vitamin B12, omega three fatty acids such as DHA and EPA, etc.

In particularly preferred embodiments, the present invention relates to the use of an algae source of DHA and/or EPA, or any other non-fish source of DHA and/or EPA as a way to improve the flavour, and compliance with treatment. This is necessary as L-MF is not soluble and needs to be suspended in a liquid. Fish sources of EPA and/or DHA are unpalatable, and not likely to be consumed as readily resulting in treatment interruptions.

In some embodiments comprise the use of encapsulation and/or micro encapsulation for some or all of the ingredients to improve the flavour, for example encapsulating fish or algal based derivatives, fishy flavour will not be present in the drink and improves long term stability by reducing risk of formation of off notes from fatty acid oxidation.

Microencapsulation is a process of building a functional barrier between the core and wall material to avoid chemical and physical reactions and to maintain the biological, functional, and physicochemical properties of core materials, in this case marine sourced DHA rich oils. Microencapsulation of marine, vegetable, and essential oils has been conducted and commercialized by employing different methods including emulsification, spray-drying, coaxial electrospray system, freeze-drying, coacervation, in situ polymerization, melt-extrusion, supercritical fluid technology, and fluidized-bed-coating. Spray-drying and coacervation are the most commonly used techniques for the microencapsulation of oils. The choice of an appropriate microencapsulation technique and wall material depends upon the end use of the product and the processing conditions involved. Microencapsulation enhances the oxidative stability, thermos-stability, shelf-life, and biological activity of oils. In addition, it can also be helpful in controlling organoleptic issues with the use of DHA due the oxidation and thus preventing sensory defects and off notes in finished products.

In some aspects of the invention, further ingredients are added in order to suspend or to enhance solubility of an active ingredient (such as L-methylfolate). Some preferred embodiments comprise a suspension of an active ingredient such as L-methylfolate calcium which is created using effervescence.

In some embodiments a preparation according to the invention is used to aid in the prevention or ameloriation of dementia and other similar disturbances. Consequently, it is important to make the preparation as easy as possible to prepare and for the user to ingest. Accordingly, in some embodiments, there is provided an effervescent form which comprises a suspension of the active ingredient, such as 5-methylfolate (eg. metafolin). In some embodiments, the preparation may comprise a dispersion of the active ingredient, however, some of these embodiments are not as organoleptically attractive and may not be preferred by users.

In some embodiments there is provided a preparation comprising an active ingredient, a suspension base and an effervescent agent. Preferably the suspension base comprises a matrix-forming component and a thickening (or viscosity) component. In some preferred embodiments, the thickening/viscosity component is provided by the presence of one or more hydrocolloids.

Hydrocolloids generally contain many hydroxyl groups and may be polyelectrolytes. Most important amongst these properties are solubility, viscosity (including thickening and gelling) and water binding but also significant are many others including emulsion stabilization, prevention of ice recrystallization and organoleptic properties. The degree with which the hydrocolloid solutions mix with saliva, determined by their degree of chain entanglement, determines flavor perception. Examples of such hydrocolloids are: Agar, Alginate, Arabinoxylan, Carrageenan, Carboxymethylcellulose, Cellulose, Curdlan, Gelatin, Gellan, β-Glucan, Guar gum, Gum Arabic, Locust bean gum, Pectin, Starch, and Xanthan gum.

In some embodiments there is provided a preparation comprising an active ingredient, a suspension base and an effervescent agent. Preferably the suspension base comprises a matrix-forming component and a thickening (or viscosity) component. In some preferred embodiments the matrix forming component comprises inulin and in some preferred embodiments the thickening (or viscosity) component comprises xanthum.

It has been surprisingly found with some particularly preferred embodiments that the effervescent agent and the effervescence thereby produced assists in mixing of the ingredients of the preparation after water has been added and thereby enhances formation of the suspension. This is important as a more complete suspension will have a greater percentage of active material caught within it and therefore available for ingestion. This is important as the active will not stay in suspension for a long period of time and it is important for the preparation to be consumed while the active is suspended so that as much as possible of the active ingredient is actually consumed. By using effervescence, the preparation not only more rapidly and completely forms the suspension, but holds it longer and is also more attractive organoleptically to the user. In some preferred embodiments there is about 5 to 40 mg, preferably 10 to 30 mg and more preferably about 15-20 mg of active ingredient per 200 ml serve, so that it is important that all of the active is consumed by the user.

It has been found that in some preferred embodiments, it is useful to use prehydrated xanthum gum as standard xanthum gum can take time to hydrate before it assists in formation of the suspension. Prehydrated xanthum acts much more quickly and thereby speeds suspension formation.

In some embodiments the thickening/viscosity component may comprise one or more of: Xanthum gum, carboxymethyl cellulose, a hydratable methyl cellulose, sodium carboxyl methyl cellulose, sodium methyl cellulose, microcrystalline cellulose group, other gums such as guar gum, pectin (if do RTD), or other hydrocolloids such as agar, carrageenan. Note that further modifications may need to be made depending on the chosen substance, for example for carrageenan, it would have to be a hot process rather than powdered.

In some embodiments, the matrix forming component may comprise any one or more of: inulin (preferred as it has very little flavour, highly soluble, has good viscosity, etc), dextrins, malto dextrins, potato dextrins (noticeable mouth feel change). Note that malto dextrins are highly soluble with a relatively low Molecular weight, so that one would need a large amount of them to achieve the same viscosity as inulin (eg. one may need 10 mg pack instead of 4 gm of inulin). Note also that malto dextrins take longer to hydrate, also pectin could possibly be used—depending on the way it is done—as it forms matrix as well as a gel—but it is not so good for powder preparation.

Other potential components may include modified starches which would provide that viscosity and suspension—maize, potato, tapeocha.

According to one preferred embodiment there is provided a composition comprising:

0.1-100 mg preferably 1 to 80 mg, preferably 10 to 50 mg, preferably 12 to 25 mg, preferably 10 to 20 mg L-methylfolate or a physiologically acceptable salt, ester or derivative thereof;
0.1 to 10 mg preferably 0.3 to 8 mg, preferably 0.5 to 5 mg, preferably 0.5 to 2 mg, preferably 0.6 to 1.2 mg Cyanocobalamin or a physiologically acceptable salt, ester or derivative thereof;
10 to 1000 mg preferably 20 to 800 mg preferably 40 to 400 mg preferably 80 to 300 mg, preferably 120 to 200 mg flavour additive which is preferably a natural flavour additive and preferably pink grapefruit flavour or the like;
80 to 8000 mg preferably 200 to 4000 mg more preferably 400 to 2000 mg, preferably 600 to 1000 mg preferably 750 to 900 mg inulin which is preferably instant inulin-fibriline;
3 to 300 mg preferably 10 to 200 mg, preferably 15 to 100 mg, preferably 25 to 50 mg, preferably 25 to 35 mg sucralose;
80 to 8000 mg preferably 200 to 4000 mg more preferably 400 to 2000 mg, preferably 600 to 1000 mg preferably 750 to 900 mg glucose syrup which is preferably glucose solids—rice;
40 to 5000 mg preferably 80 to 3000 mg, preferably 100 to 1000 mg, preferably 200 to 800 mg, preferably 400 to 600 mg sodium carbonate;
150 to 15,000 mg preferably 300 to 10,000 mg, preferably 500 to 5000 mg, preferably 1000 to 2000 mg, preferably 1200 to 1700 mg citric acid anhydrous;
1 to 200 mg preferably 1 to 100 mg preferably 1 to 50 mg, preferably 1 to 30 mg, preferably 10 to 22 mg natural colour;
1 to 200 mg preferably 1 to 100 mg preferably 1 to 50 mg, preferably 1 to 30 mg, preferably 10 to 22 mg xanthan gum 200 mesh;
40 to 5000 mg preferably 80 to 3000 mg, preferably 100 to 1000 mg, preferably 200 to 800 mg, preferably 450 to 650 mg sodium bicarbonate.

In some embodiments of the invention, there is provided a method of ameliorating or reducing the clinical signs of a brain-related condition comprising administering such a composition once a day. In some preferred embodiments, the brain-related condition is depression or a depression-related condition.

According to one preferred embodiment there is provided a composition comprising:

0.1 to 10 mg preferably 0.3 to 8 mg, preferably 0.5 to 5 mg, preferably 0.8 to 4 mg, preferably 1.5 to 3 mg L-methylfolate or a physiologically acceptable salt, ester or derivative thereof;
0.1 to 10 mg preferably 0.3 to 8 mg, preferably 0.5 to 5 mg, preferably 0.9 to 3.5 mg, preferably 1.5 to 3 mg Cyanocobalamin or a physiologically acceptable salt, ester or derivative thereof;
0.1-200 mg preferably 1 to 120 mg, preferably 10 to 100 mg, preferably 20 to 80 mg, preferably 40 to 60 mg Vitamin B6 or a physiologically acceptable salt, ester or derivative thereof;
50 to 15,000 mg optionally 150 to 10,000 mg, optionally 500 to 5000 mg, optionally 1000 to 2000 mg, optionally 1500 to 2500 mg omega three fatty acid which optionally comprises docosahexaenoic acid (DHA) or eicosapentaenoic acid (EPA) or a mixture thereof;
10 to 1000 mg preferably 20 to 800 mg preferably 40 to 400 mg preferably 80 to 300 mg, preferably 120 to 200 mg flavour additive which is preferably a natural flavour additive and preferably pink grapefruit flavour or the like;
80 to 8000 mg preferably 200 to 4000 mg more preferably 400 to 2000 mg, preferably 600 to 1000 mg preferably 750 to 900 mg inulin which is preferably instant inulin-fibriline;
3 to 300 mg preferably 10 to 200 mg, preferably 15 to 100 mg, preferably 25 to 50 mg, preferably 25 to 35 mg sucralose;
80 to 8000 mg preferably 200 to 4000 mg more preferably 400 to 2000 mg, preferably 600 to 1000 mg preferably 750 to 900 mg glucose syrup which is preferably glucose solids—rice;
40 to 5000 mg preferably 80 to 3000 mg, preferably 100 to 1000 mg, preferably 200 to 800 mg, preferably 400 to 600 mg sodium carbonate;
150 to 15,000 mg preferably 300 to 10,000 mg, preferably 500 to 5000 mg, preferably 1000 to 2000 mg, preferably 1200 to 1700 mg citric acid anhydrous;
1 to 200 mg preferably 1 to 100 mg preferably 1 to 50 mg, preferably 1 to 30 mg, preferably 10 to 22 mg natural colour;
1 to 200 mg preferably 1 to 100 mg preferably 1 to 50 mg, preferably 1 to 30 mg, preferably 10 to 22 mg xanthan gum 200 mesh;
40 to 5000 mg preferably 80 to 3000 mg, preferably 100 to 1000 mg, preferably 200 to 800 mg, preferably 450 to 650 mg sodium bicarbonate.

In some embodiments of the invention, there is provided a method of ameliorating or reducing the clinical signs of a brain-related condition comprising administering such a composition once a day.

In some preferred embodiments, the brain-related condition is dementia or a dementia-related condition. During two years of treatment in Mild Cognitive Impairment (MCI) patients, Smith et al found strong evidence that B vitamin treatment significantly modified disease progression (The Journal of Prevention of Alzheimer's Disease (2017) Vol 3, pages 1-3.

In some preferred embodiments, the brain-related condition is depression or a depression-related condition. In two multicentre sequential parallel comparison design trials, Papakosatas et al concluded that adjunctive L-methylfolate at 15 mg/day may constitute an effective safe and relatively well tolerated treatment strategy for patients with major depressive disorder who have a partial responses or no response to SSRIs (Am J Psychiatry 2012; 169:1267-1274).

It will be appreciated by the skilled addressee that salts or esters of the compounds comprising formulations of the invention may also be used. For example, in some preferred embodiments, the calcium salt of L-methylfolate is preferred.

### Example 1

Metafolin (L-Methylfolate Calcium) beverage for dietary treatment of dementia and depression.

Description:

A powdered formulation designed to produce a pleasant flavoured effervescing fortified beverage containing Metafolin and other fortifying vitamins.

A powdered formulated beverage mixed with potable tap water, stirred and consumed for the treatment of depression or dementia in the form of a substantially optically clear solution, with light natural colours, may be effervescing or still, wherein a 200 ml servicing comprises:
Between about 1 mg and about 50 mg of L-Methylfolate calcium—the main biologically active compound in the powdered beverage.
Quantities of vitamin B12 (cyanobalamin) and/or vitamin B6 (pyridoxine hydrochloride), wherein each quantity is between 10% and about 500% of recommended daily value as supporting biologically active compounds.
Quantities of dry glucose powder as a dispersing carbohydrate source at between 5%-25% dry matter basis providing dispersion of the active ingredients as well as sweetness and body to the beverage.
Quantities of natural flavouring ingredients at between 0.1 and 5.0% on a dry matter basis for the purpose of imparting a specific taste to the final beverage.
Quantities of inulin (fructo-oligosachharides) at between 1% and 50% on a dry matter basis for the purpose of improving dispersion of the active ingredients and imparting a slightly thickened mouthfeel for the purpose of improved organoleptic perception.
Quantities of polysaccharide gums including xanthum gum or guar gum or a combination of both at between 0% and 10% of the product on a dry matter basis for the purpose of suspension and even distribution and prevention of settling of the active ingredients in the final beverage.
Quantities of intense sweetener, being either sucralose or asulfume-K, or Neotame or a combination of those intense sweeteners for the purpose of sweetening the beverage from 0.01% to 1% on a dry matter basis.
Quantities of sodium carbonate, sodium bicarbonate and citric acid to act as an effervescing agent on contact with potable water in the beverage to impart a pleasant lightly carbonated beverage, individually or in combination at between 10% and 50% on a dry matter basis.

### Example 2

Two example formulations according to one aspect of the invention, include:

| | | Concentration | |
|---|---|---|---|
| Formulation | Ingredients | % w/v | Observations |
| Product 1 | L-methylfolate calcium | 0.016 | Effervescence effect persisted for 25 s |
| | Cyanocobolamin (Vit B12) | 0.001 | Produced a cloudy pink solution. |
| | Lactose monohydrate | 2.305 | A few particles settled to the bottom of the |
| | Natural orange flavour | 0.073 | beaker - easily re-dispersed upon stirring |
| | powder | | of the solution. |
| | Citric acid | 0.856 | Smells like oranges. |
| | Sodium bicarbonate | 0.724 | |
| | Sucralose | 0.026 | |
| Product 2 | L-methylfolate calcium | 0.006 | Effervescence effect persisted for 20 s |
| | Cyanocobolamin (Vit B12) | 0.001 | Produced a cloudy light pink solution. |
| | Pyridoxine hydrochloride | 0.020 | A few particles (dark pink in colour) settled |
| | (Vit B9) | | to the bottom of the beaker, however are |
| | Lactose monohydrate | 2.299 | easily re-dispersed upon stirring of the |
| | Natural orange flavour | 0.0723 | solution. |
| | powder | | Smells like oranges. |
| | Citric acid | 0.854 | |
| | Sodium bicarbonate | 0.722 | |
| | Sucralose | 0.026 | |

### Product 1

| | |
|---|---|
| Ingredients | Mass (g) |

| | | |
|---|---|---|
| L-methylfolate calcium | 0.0150 | 0.0750 |
| Cyanocobalamin (Vitamin B12) | 0.0010 | 0.0050 |
| Bulking agent - lactose monohydrate | 2.2469 | 11.2345 |
| Citric acid | 0.8347 | 4.1734 |
| Sodium bicarbonate | 0.7061 | 3.5306 |
| Trusil natural orange flavour powder | 0.0707 | 0.3534 |
| Thickener-MHEC | 0.1000 | 0.5000 |
| Sucralose | 0.0256 | 0.1282 |
| Total mass of powder (g) | 4 | 20 |

### Product 2

| | |
|---|---|
| Ingredients | Mass (g) |

| | | |
|---|---|---|
| L-methylfolate calcium | 0.0056 | 0.028 |
| Cyanocobalamin (Vitamin B12) | 0.0010 | 0.005 |
| Pyridoxine Hydrochloride (Vitamin B6) | 0.0200 | 0.1 |
| Bulking agent - lactose monohydrate | 2.2408 | 11.20381 |
| Citric acid | 0.8324 | 4.1619683 |
| Sodium bicarbonate | 0.7042 | 3.5209206 |
| Trusil natural orange flavour powder | 0.0705 | 0.3524794 |
| Sucralose | 0.0256 | 0.1278222 |
| Thickener-MHEC | 0.1000 | 0.5 |
| Total mass of powder (g) | 4 | 20 |

In some embodiments, the formulations may additionally comprise a small amount of MHEC to reduce sedimentation. For example, this may be less than or equal to 2%, preferably less than 1% and in some preferred embodiments, about 0.1%.

In some embodiments, the formulation comprises no sucralose and in some embodiments, lactose is replaced with another compound, such as mannitol or sorbitol. In some embodiments, the formulation further comprises sodium chloride to balance sweet and salt taste. In some embodiments, there is provided an alternative flavour, such as strawberry, raspberry, cherry, etc.

### Example 3

A further example preparation (for a 30 kg batch size of 4.5 gm sachets) is as follows:

| | | Kg/ | | Grams per |
|---|---|---|---|---|
| Ingredients | Suppliers | blend | % | serve |
| L-methylfolate | Merck | 0.105 | 0.351 | 0.01581 |
| Cyanocobalamin | Hellay | 0.008 | 0.026 | 0.00117 |
| Pink Grapefruit | Tastemaster | 1.110 | 3.699 | 0.16647 |
| flavour, natural | | | | |
| Instant inulin- | Brenning Aust | 5.550 | 18.500 | 0.83251 |
| Fibriline | | | | |
| Sucralose | Ingredient | 0.200 | 0.666 | 0.02996 |
| | Techniques | | | |
| | Australia | | | |
| Glucose Syrup | Agrifoods | 5.600 | 18.667 | 0.84001 |
| Solids - Rice | | | | |
| Sodium | Redox | 3.300 | 11.00 | 0.49500 |
| Carbonate (Soda | | | | |
| Ash) | | | | |
| Citric Acid | Redox | 10.000 | 33.334 | 1.50001 |
| anhydrous | | | | |
| Natural Colour - | Hawkins Watts | 0.111 | 0.370 | 0.01665 |
| Safflower Extract | | | | |
| Xanthan Gum | Ticaxan/Tic | 0.111 | 0.370 | 0.01665 |
| 200 mesh | Gums | | | |
| Sodium | Redox | 3.905 | 13.017 | 0.58576 |
| Bicarbonate | | | | |
| TOTAL | | 30.000 | 100.000 | 4.500 |

The product is a free flowing fortified powdered drink. It is made up in water and provides a lightly effervescent pink grapefruit flavoured and coloured drink with slight acidity and fruity sweetness.

To serve—add entire sachet to 200 ml of plain water (may be chilled), stir vigorously for 30 seconds and drink immediately.

To store—keep below 25° C. in a cool dark place away from direct sun light.

Nutritional Information:

NUTRITION INFORMATION
Servings per package: 1
Serving size: 4.5 g

| | |
|---|---|
| Average Quantity | Average Quantity |
| per Sewing | per 100 g |

| | | |
|---|---|---|
| Energy | 36 kJ (9 Cal) | 794 kJ (190 Cal) |
| Protein | less than 0.1 g | less than 0.1 g |
| Gluten | Not Detected | Not Detected |
| Fat, Total | less than 0.1 g | less than 0.1 g |
| Saturated | less than 0.1 g | less than 0.1 g |

| | | | | |
|---|---|---|---|---|
| Carbohydrate | 1.0 | g | 22.1 | g |
| Sugars | 0.2 | g | 4.4 | g |
| Lactose | 0.0 | g | 0.0 | g |
| Galactose | 0.0 | g | 0.0 | g |
| Sodium | 396 | mg | 8790 | mg |
| L-Methyitolate | 15 | mg | 333 | mg |
| Cyanocobslamin (B12) | 1 | mg | 22 | mg |

Manufacture:

### Pre-Production Preparation

1. Pre weighed vitamins are blended with 5 kg of rice glucose syrup solids to make a 100% Vitamin premix for adding to the final formulation to improve mixing

### Production

2. All other raw materials are batched and blended in 30 kg batches with samples taken and tested in the lab for consistency and even distribution of blended raw materials, product must be screened through final screen of <3 mm.
3. Samples are to be taken and compared against retained approved samples for flavour and colour to ensure homogeneous blending, if samples do not match then blending is to continue until samples are acceptable.
4. 20 g of the final blended product shall be retained as a reference sample from production—this shall be hermetically sealed in appropriate high barrier packaging and stored away from light, heat and volatile materials and labelled with the product name, batch code and best before date (use of final carton label acceptable for traceability).
5. Once passed by QA the blended materials are taken to the filling room and placed in the filling hopper.
6. Product can now be filled into sachets—pre-printed film of the correct variety is to be used for each product to 4.5 g±0.25 g, sealed, date and batch coded and is metal detected prior to final packing.
7. Seals to be checked every 15 minutes, if any defects are found all product produced from the proceeding 15 minutes are to be isolated and individually inspected, any products found to have faulty seals are to be rejected (controlled unpacking of the rejected sachets may be reworked)
8. Products are packed into cartons.

### Example 4

A further example preparation (for a 30 kg batch size of 4.5 gm sachets) is as follows:

| | Kg/ | | Grams per |
|---|---|---|---|
| Ingredient | blend | % | serve |
| L-methylfolate | 0.118 | 0.39 | 0.018 |
| Cyanocobalamin | 0.08 | 0.03 | 0.001 |
| Pink grapefruit flavour, | 1.110 | 3.70 | 0.166 |
| natural TM767873 | | | |
| Instant Inulin-Fibriline | 5.550 | 18.50 | 0.832 |
| Sucralose | 0.200 | 0.67 | 0.030 |
| Glucose Syrup Solids - Rice | 5.600 | 18.67 | 0.840 |
| Sodium Carbonate (Soda Ash) | 3.300 | 11.00 | 0.495 |
| Citric Acid anhydrous | 10.000 | 33.33 | 1.500 |
| Natural Colour-Safflower | 0.111 | 0.37 | 0.017 |
| Extract Plant Ex | | | |
| CFO13905A | | | |
| Xanthan Gum 200 mesh | 0.111 | 0.37 | 0.017 |
| Sodium Bicarbonate | 3.893 | 12.98 | 0.584 |
| Total | 30.000 | 100.00 | 4.500 |

This formulation is preferred for uses relevant to ameliorating depression in a subject suffering therefrom.

One method of treatment is to add the entire sachet to 200 ml plain water, stir vigorously for 3 seconds and drink immediately.

The product is a free flowing fortified powdered drink. It is made up in water and provides a lightly effervescent pink grapefruit flavoured and yellow to slightly pink coloured drink with slight acidity and fruity sweetness.

Nutritional Information

Servings per package: 1
Serving size: 4.5 g

| | |
|---|---|
| Average Quantity | Average Quantity |
| per Serving | per 100 g |

| | | |
|---|---|---|
| Energy | 36 kJ (9 Cal) | 794 kJ (190 Cal) |
| Protein | Less than 0.1 g | Less than 0.1 g |
| Gluten | Not Detected | Not Detected |
| Fat, Total | Less than 0.1 g | Less than 0.1 g |
| Saturated | Less than 0.1 g | Less than 0.1 g |

| | | | | |
|---|---|---|---|---|
| Carbohydrate | 1.0 | g | 22.1 | g |
| Sugars | 0.2 | g | 4.4 | g |
| Lactose | 0.0 | g | 0.0 | g |
| Galactose | 0.0 | g | 0.0 | g |
| Sodium | 396 | mg | 8790 | mg |
| L-methylfolate | 15 | mg | 333 | mg |
| Calcium | | | | |
| Cyanocobalamin | 1 | mg | 22 | mg |
| (B12) | | | | |

This embodiment of the invention contains no artificial colours or flavours and no preservatives, it is free of gluten, dairy, peanuts and lactose.

### Example 5

A further example preparation (for a 30 kg batch size of 6.55 gm sachets) is as follows:

| | Kg/ | | Grams per |
|---|---|---|---|
| Ingredient | blend | % | serve |
| L-methylfolate Calcium | 0.011 | 0.04 | 0.0025 |
| Cyanocobalamin | 0.009 | 0.03 | 0.0020 |
| Vitamin B6 | 0.230 | 0.77 | 0.0500 |
| *Omega-3 fatty acids | 9.181 | 30.60 | 2.0000 |
| Pink grapefruit flavour, | 0.762 | 2.54 | 0.1660 |
| natural TM767873 | | | |
| Instant Inulin-Fibriline | 3.819 | 12.73 | 0.8320 |
| Sucralose | 0.138 | 0.46 | 0.0300 |
| Glucose Syrup Solids - Rice | 3.856 | 12.85 | 0.8400 |
| Sodium Carbonate (Soda Ash) | 2.272 | 7.57 | 0.4950 |
| Citric Acid anhydrous | 6.885 | 22.95 | 1.5000 |
| Natural Colour-Safflower | 0.078 | 0.26 | 0.0170 |
| Extract Plant Ex | | | |
| CFO13905A | | | |
| Xanthan Gum 200 mesh | 0.078 | 0.26 | 0.0170 |
| Sodium Bicarbonate | 2.681 | 8.94 | 0.5840 |
| TOTAL | 30 | 100 | 6.5355 |

*Omega 3 fatty acids can be chosen from any suitable acids, but are preferably chosen from docosahexaenoic acid (DHA) or eicosapentaenoic acid (EPA) or a mixture thereof.

This formulation is preferred for uses relevant to ameliorating dementia in a subject suffering therefrom. One method of treatment is to add the entire sachet to 200 ml plain water, stir vigorously for 3 seconds and drink immediately.

The product is a free flowing fortified powdered drink. It is made up in water and provides a lightly effervescent pink grapefruit flavoured and yellow to slightly pink coloured drink with slight acidity and fruity sweetness.

This embodiment of the invention contains no artificial colours or flavours and no preservatives, it is free of gluten, dairy, peanuts and lactose.

### Example 6

An example manufacturing process according to the invention.

### Pre-Production Preparation

1. Pre-weighed vitamins are blended with rice glucose syrup solids to make a 100% Vitamin premix for adding to the final formulation to improve mixing.
2. Production room to be held at a managed relative humidity of 20-35% ERH Maximum ERH 40%.
3. If ERH of packing room is above 40% then product is to be removed from the blending or packing rooms and sealed in Airtight containers until the ERH has been reduced to below 35%.

### Production

4. All other raw materials are batched and blended in 30 kg batchesd with samples taken and tested in the laboratory for consistency and even distribution of blended raw materials.
5. Samples are to be taken and compared against retained approved samples for flavour and colour to ensure homogenous blending. If samples do not match then blending is to continue until samples are acceptable.
6. Product muse be screened through final screen of <3 mm.
7. Once passed by QA the blended materials are taken to the filling room and placed in the filling hopper.
8. Product can be filled into sachets—pre-printed film of the correct variety is to be used for each product to 4.5+/−0.25 g, sealed, date and batch coded and is x-ray detected prior to final packaging.
9. Seals to be checked every 15 minutes, if any defects are found, all product produced from the preceding 15 minutes are to be isolated and individually inspected. Any product found to have faulty seals are to be rejected.
10. Products are packed into cartons, sachets are to be packed into pre-printed lithographically printed solid fibre cartons at 30×4.5 g satchets, closed with tamper evident dart seal and date and batch coded.
11. The packs are then placed in shippers×6 and a carton date coded as specified.
12. Cartons are palletized to the pallet configuration provided, stretch wrapped and layer pad placed on the top of each full pallet to reduce the chance of dust ingress. Pallet labels are to be attached to each pallet identifying each pallet from production by batch date and best before date.′

### Example 7

A further example preparation (for a 30 kg batch size of 4.5 gm sachets) is as follows:

| | Kg/ | | Grams per |
|---|---|---|---|
| Ingredient | blend | % | serve |
| L-methylfolate | 0.118 | 0.39 | 0.018 |
| Pink grapefruit flavour, | 1.110 | 3.70 | 0.166 |
| natural TM767873 | | | |
| Instant Inulin-Fibriline | 5.558 | 18.53 | 0.831 |
| Sucralose | 0.200 | 0.67 | 0.030 |
| Glucose Syrup Solids - Rice | 5.600 | 18.67 | 0.840 |
| Sodium Carbonate (Soda Ash) | 3.300 | 11.00 | 0.495 |
| Citric Acid anhydrous | 10.000 | 33.33 | 1.500 |
| Natural Colour-Safflower | 0.111 | 0.37 | 0.017 |
| Extract Plant Ex | | | |
| CFO13905A | | | |
| Xanthan Gum 200 mesh | 0.111 | 0.37 | 0.017 |
| Sodium Bicarbonate | 3.893 | 12.98 | 0.584 |
| Total | 30.000 | 100.00 | 4.500 |

This formulation is preferred for uses relevant to ameliorating depression in a subject suffering therefrom.

One method of treatment is to add the entire sachet to 200 ml plain water, stir vigorously for 3 seconds and drink immediately.

The product is a free flowing fortified powdered drink. It is made up in water and provides a lightly effervescent pink grapefruit flavoured and yellow to slightly pink coloured drink with slight acidity and fruity sweetness.

### Example 8

A further example preparation (for a 30 kg batch size of 6.55 gm sachets) is as follows:

| Ingredient | amount per serve | |
|---|---|---|
| L-methylfolate Calcium | 1.1 to 8 mg | |
| Cyanocobalamin | 0.0089 | mg |
| Vitamin B6 | 0.89 | mg |
| DHA | 100 to 700 mg | |
| EPA | variable | |
| Pink grapefruit flavour, natural TM767873 | 0.1660 | gm |
| Instant Inulin-Fibriline | 0.8320 | gm |
| Sucralose | 0.0300 | gm |
| Glucose Syrup Solids - Rice | 0.8400 | gm |
| Sodium Carbonate (Soda Ash) | 0.4950 | gm |
| Citric Acid anhydrous | 1.5000 | gm |
| Natural Colour-Safflower Extract Plant Ex | 0.0170 | gm |
| CFO13905A | | |
| Xanthan Gum 200 mesh | 0.0170 | gm |
| Sodium Bicarbonate | 0.5840 | gm |

| | |
|---|---|
| Trehalose | variable |

This formulation is preferred for uses relevant to ameliorating dementia in a subject suffering therefrom. One method of treatment is to add the entire sachet to 200 ml plain water, stir vigorously for 3 seconds and drink immediately.

The product is a free flowing fortified powdered drink. It is made up in water and provides a lightly effervescent pink grapefruit flavoured and yellow to slightly pink coloured drink with slight acidity and fruity sweetness.

This embodiment of the invention contains no artificial colours or flavours and no preservatives, it is free of gluten, dairy, peanuts and lactose.

### Example 9

A further example preparation (for a 30 kg batch size of 6.55 gm sachets) is as follows:

| Ingredient | amount per serve | |
|---|---|---|
| L-methylfolate Calcium | 1.1 to 8 mg | |
| Cyanocobalamin | 2.21 | mg |
| Vitamin B6 | 44.1 | mg |
| DHA | 100 to 700 mg | |
| EPA | variable | |
| Pink grapefruit flavour, natural TM767873 | 0.1660 | gm |
| Instant Inulin-Fibriline | 0.8320 | gm |
| Sucralose | 0.0300 | gm |
| Glucose Syrup Solids - Rice | 0.8400 | gm |
| Sodium Carbonate (Soda Ash) | 0.4950 | gm |
| Citric Acid anhydrous | 1.5000 | gm |
| Natural Colour-Safflower Extract Plant Ex | 0.0170 | gm |
| CFO13905A | | |
| Xanthan Gum 200 mesh | 0.0170 | gm |
| Sodium Bicarbonate | 0.5840 | gm |

| | |
|---|---|
| Trehalose | variable |

This formulation is preferred for uses relevant to ameliorating dementia in a subject suffering therefrom. One method of treatment is to add the entire sachet to 200 ml plain water, stir vigorously for 3 seconds and drink immediately.

The product is a free flowing fortified powdered drink. It is made up in water and provides a lightly effervescent pink grapefruit flavoured and yellow to slightly pink coloured drink with slight acidity and fruity sweetness.

This embodiment of the invention contains no artificial colours or flavours and no preservatives, it is free of gluten, dairy, peanuts and lactose.

### Example 10

A further example preparation (for a 30 kg batch size of 6.55 gm sachets) is as follows:

| Ingredient | amount per serve | |
|---|---|---|
| L-methylfolate Calcium | 1.1 to 8 mg | |
| Cyanocobalamin | 2.21 | mg |
| Vitamin B6 | 0.89 | mg |
| DHA | 100 to 700 mg | |
| EPA | variable | |
| Pink grapefruit flavour, natural TM767873 | 0.1660 | gm |
| Instant Inulin-Fibriline | 0.8320 | gm |
| Sucralose | 0.0300 | gm |
| Glucose Syrup Solids - Rice | 0.8400 | gm |
| Sodium Carbonate (Soda Ash) | 0.4950 | gm |
| Citric Acid anhydrous | 1.5000 | gm |
| Natural Colour-Safflower Extract Plant Ex | 0.0170 | gm |
| CFO13905A | | |
| Xanthan Gum 200 mesh | 0.0170 | gm |
| Sodium Bicarbonate | 0.5840 | gm |

| | |
|---|---|
| Trehalose | variable |

This formulation is preferred for uses relevant to ameliorating dementia in a subject suffering therefrom. One method of treatment is to add the entire sachet to 200 ml plain water, stir vigorously for 3 seconds and drink immediately.

The product is a free flowing fortified powdered drink. It is made up in water and provides a lightly effervescent pink grapefruit flavoured and yellow to slightly pink coloured drink with slight acidity and fruity sweetness.

This embodiment of the invention contains no artificial colours or flavours and no preservatives, it is free of gluten, dairy, peanuts and lactose.

### Example 11

A further example preparation (for a 30 kg batch size of 6.55 gm sachets) is as follows:

| Ingredient | amount per serve | |
|---|---|---|
| L-methylfolate Calcium | 1.1 to 8 mg | |
| Cyanocobalamin | 0.0089 | mg |
| Vitamin B6 | 44.1 | mg |
| DHA | 100 to 700 mg | |
| EPA | variable | |
| Pink grapefruit flavour, natural TM767873 | 0.1660 | gm |
| Instant Inulin-Fibriline | 0.8320 | gm |
| Sucralose | 0.0300 | gm |
| Glucose Syrup Solids - Rice | 0.8400 | gm |
| Sodium Carbonate (Soda Ash) | 0.4950 | gm |
| Citric Acid anhydrous | 1.5000 | gm |
| Natural Colour-Safflower Extract Plant Ex | 0.0170 | gm |
| CFO13905A | | |
| Xanthan Gum 200 mesh | 0.0170 | gm |
| Sodium Bicarbonate | 0.5840 | gm |

| | |
|---|---|
| Trehalose | variable |

This formulation is preferred for uses relevant to ameliorating dementia in a subject suffering therefrom. One method of treatment is to add the entire sachet to 200 ml plain water, stir vigorously for 3 seconds and drink immediately.

The product is a free flowing fortified powdered drink. It is made up in water and provides a lightly effervescent pink grapefruit flavoured and yellow to slightly pink coloured drink with slight acidity and fruity sweetness.

This embodiment of the invention contains no artificial colours or flavours and no preservatives, it is free of gluten, dairy, peanuts and lactose.

### Example 12

Two further examples which provide example amounts of various ingredients of formulations according to the invention.

| | Example 12a | Example 12b |
|---|---|---|
| B6 | 0.89 mg | 44.1 mg |
| B9 (FA | 1.1, 2, 3, 4, | 1.1, 2, 3, 4, |
| or LMF) | 5, 6, 7 or 8 mg | 5, 6, 7 or 8 mg |
| B12 | 0.0089 mg | 2.21 |
| DHA | 100, 200, 300, 400, | 100, 200, 300, 400, |
| | 500, 600, 700 mg | 500, 600, 700 mg |
| EPA | Undefined | Undefined |
| Trehalose | Undefined | Undefined |

While the invention has been particularly shown and described with reference to various embodiments, it will be recognized by those skilled in the art that modifications and changes may be made to the present invention without departing from the spirit and scope thereof. The scope of the invention should therefore be determined not with reference to the above description but with reference to the appended claims along with their full scope of equivalents.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

What is claimed is:

1.-22. (canceled)

23. An effervescent formulation for ameliorating or reducing the signs of a brain-related condition, comprising at least one active agent, at least one pharmaceutically acceptable effervescent acid, at least one effervescent base and at least another excipient wherein the active agent comprises L-methylfolate.

24. The composition for ameliorating or reducing the signs of a brain-related condition according to claim 23, comprising: L-methylfolate or a physiologically acceptable salt, or ester thereof; flavour additive; inulin; sucralose; a sweetener; sodium carbonate; citric acid anhydrous; xanthan gum; sodium bicarbonate.

25. The composition according to claim 24, comprising Cyanocobalamin or a physiologically acceptable salt or ester thereof.

26. The composition according to claim 24, comprising:
0.1-100 mg L-methylfolate or a physiologically acceptable salt, or ester thereof;
80 to 8000 mg inulin;
3 to 300 mg sucralose;
glucose;
40 to 5000 mg sodium carbonate;
150 to 15,000 mg citric acid anhydrous;
1 to 200 mg xanthan gum 200 mesh;
40 to 5000 mg sodium bicarbonate.

27. The composition according to claim 24, comprising: 10 to 20 mg L-methylfolate or a physiologically acceptable salt, ester or derivative thereof.

28. The composition according to claim 24, comprising: 750 to 900 mg inulin.

29. The composition according to claim 24, comprising: 25 to 35 mg sucralose.

30. The composition according to claim 24, comprising: 750 to 900 mg glucose.

31. The composition according to claim 24, comprising: 400 to 600 mg sodium carbonate.

32. The composition according to claim 24, comprising: 1200 to 1700 mg citric acid anhydrous.

33. The composition according to claim 24, comprising: 10 to 22 mg xanthan gum 200 mesh.

34. The composition according to claim 24, comprising: 450 to 650 mg sodium bicarbonate.

35. The composition according to claim 24, comprising:
10 to 20 mg L-methylfolate or a physiologically acceptable salt, ester or derivative thereof;
120 to 200 mg flavour additive;
750 to 900 mg inulin;
25 to 35 mg sucralose;
750 to 900 mg glucose;
400 to 600 mg sodium carbonate;
1200 to 1700 mg citric acid anhydrous;
10 to 22 mg natural colour;
10 to 22 mg xanthan gum 200 mesh;
450 to 650 mg sodium bicarbonate.

36. The method of ameliorating or reducing the clinical signs of a brain-related condition comprising administering a preparation or composition according to any one of the preceding claims once per day.
